(19) United States
(12) Patent Application Publication
(10) Pub. No.: US 2025/0339628 A1
(43) Pub. Date: Nov. 6, 2025

(54) METHODS AND SYSTEMS FOR DELIVERING FORMULATIONS TO USERS USING MODULAR DEVICE HAVING REMOVABLE CARTRIDGE

(71) Applicant: MICRONEB TECH HOLDINGS, INC., ST PETERSBURG, FL (US)

(72) Inventors: Pradeep Albert, M.D., Sarasota, FL (US); Christine Nichols, Largo, FL (US); David J. Condron, Seminole, FL (US); Brian Artze, Gulfport, FL (US); Fadi Saba, M.D., St Petersburg, FL (US); Jesse Klein, D.O., Clearwater, FL (US); Vijay Vad, M.D., New York, NY (US); Shawn Best, Tampa, FL (US)

(21) Appl. No.: 18/946,604

(22) Filed: Nov. 13, 2024

Related U.S. Application Data
(63) Continuation-in-part of application No. 18/654,471, filed on May 3, 2024, now Pat. No. 12,156,966.

(51) Int. Cl. A61M 11/00; A61M 39/24

(52) U.S. Cl. CPC A61M 11/005; A61M 39/24; A61M 2202/0007; A61M 2205/0233; A61M 2205/121; A61M 2205/3306; A61M 2205/3317; A61M 2205/3327; A61M 2205/3331; A61M 2205/50; A61M 2205/583; A61M 2209/088

(57) ABSTRACT

The present invention relates to a formulation delivery system designed to deliver formulations through a removable cartridge that houses an atomizer and wick assembly. The system is adaptable for multiple medications and environments, including wearable devices such as masks, helmets, and protective suits. The removable cartridge ensures controlled, consistent delivery of medications, including solutions, suspensions, and emulsions. Key features include air-tight seals, sensor technology for cartridge detection and dosage control, and a modular design that allows easy replacement or refilling. This system addresses the challenges of traditional nebulizers by improving portability, ease of use, and precision. It is particularly suited for patients with chronic respiratory conditions or emergency situations where rapid medication delivery, such as naloxone for opioid overdoses, is crucial. The system is also integrated with modern technologies, offering real-time monitoring and feedback to ensure user safety and compliance.

### REFERENCE TO RELATED APPLICATIONS

This application is a continuation in part application of U.S. Non-Provisional application Ser. No. 18/654,471 titled “Methods and Systems for Delivering Formulations to Users Using Modular Device Having Removable Cartridge” and filed May 3, 2024, the subject matter of which is hereby incorporated by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The present disclosure relates to the field of mesh nebulizers, and more specifically to the field of mesh nebulizers for administering medications.

### BACKGROUND

The delivery of medications through inhalation has long been a cornerstone in the treatment of respiratory diseases such as asthma, chronic obstructive pulmonary disease (COPD), and cystic fibrosis. These conditions often require that patients regularly inhale medications directly into their lungs, providing relief from symptoms and, in some cases, preventing the progression of the disease. Traditional nebulizers and inhalers have been the primary devices used for this purpose. While they have proven effective in many respects, there remain significant challenges that limit their overall effectiveness and ease of use.

One of the main limitations of traditional nebulizers, particularly jet nebulizers, is their bulky size and dependence on a power source, which significantly limits portability. Additionally, they are often noisy, which can make them impractical in certain settings. These factors have led to the development of more advanced devices, such as vibrating mesh nebulizers, which are quieter, smaller, and more portable. Vibrating mesh nebulizers operate by forcing liquid medication through a fine mesh with tiny holes, which produces an aerosol mist that can be easily inhaled by the patient. However, while these devices are an improvement over traditional models, they still come with their own set of challenges, including the potential for clogging in the mesh and the need for meticulous cleaning and maintenance to ensure continued efficiency.

A critical aspect of inhalation-based drug delivery is the precise control over medication dosage. This is particularly important for medications that require exact dosages to be effective, such as bronchodilators, steroids, or biologics used in severe cases of asthma or other chronic respiratory conditions. Inconsistent dosage delivery can result in under-treatment, where the patient does not receive enough medication to alleviate symptoms, or over-treatment, which can lead to side effects or complications. Traditional systems often lack the sophistication needed to ensure that each dose is delivered consistently and accurately, leading to variability in treatment outcomes.

Furthermore, patient compliance is another significant issue in respiratory care. The correct use of nebulizers and inhalers requires a certain level of patient knowledge and dexterity. For example, metered-dose inhalers (MDIs) require the patient to coordinate the act of inhalation with the release of medication, which can be difficult, particularly for young children, the elderly, or those with severe respiratory conditions. Even with dry powder inhalers (DPIs), which do not require chemical propellants, the patient must inhale forcefully enough to ensure the medication reaches deep into the lungs. User errors, such as incorrect technique or improper device maintenance, can drastically reduce the effectiveness of these treatments.

Beyond these common use cases, there is a growing demand for versatile drug delivery systems that can accommodate emerging pharmaceutical needs, such as the delivery of biologics or drugs used in emergency situations. For instance, the ongoing opioid crisis has heightened the need for devices that can quickly and efficiently administer life-saving medications like naloxone, which reverses the effects of opioid overdose. Naloxone must be delivered rapidly to prevent respiratory failure and other life-threatening symptoms. Existing systems for naloxone delivery, such as auto-injectors and nasal sprays, are effective but often require significant user intervention, which can introduce delays in emergency situations where every second counts.

Another emerging public health concern is the contamination of illicit drugs with xylazine, a veterinary sedative that can cause profound sedation and respiratory depression when consumed by humans. In such cases, naloxone is often ineffective, and there is increasing interest in using drugs like yohimbine, an alpha-2 adrenergic receptor antagonist, to counteract xylazine's effects. However, current delivery systems are not designed to accommodate these rapidly evolving needs, underscoring the need for more flexible, adaptable devices.

In addition to the specific therapeutic challenges posed by these medications, there are broader issues related to the practicality and usability of current drug delivery systems. Many traditional systems are not designed with portability in mind, which can be problematic for patients who need to carry their medication with them throughout the day. For example, a person suffering from asthma may need to use their nebulizer multiple times a day, but carrying a bulky device is impractical in many situations. Furthermore, systems that require frequent refills or complex assembly can lead to user frustration and non-compliance. Similarly, the risk of contamination during the refilling or handling process is a constant concern, particularly for medications that must remain sterile.

Another significant challenge in prior inhalation delivery systems is the lack of modularity and interchangeability. Many devices are designed to deliver only a specific medication, limiting their versatility. For example, a patient using one type of nebulizer for asthma may need to switch to a completely different system to administer another type of medication. This lack of flexibility not only increases the burden on patients but also raises costs, as patients may need to purchase and maintain multiple devices. In modern healthcare, there is an increasing push towards universal or interchangeable systems that can be used across a range of medications, simplifying treatment regimens, and reducing costs.

Furthermore, there is a need for drug delivery systems that can integrate with modern technologies, such as sensors and connectivity features, which enable real-time monitoring of medication usage, dosage control, and patient compliance. With the advent of smart medical devices, it is possible to imagine a future where patients can receive personalized treatments based on their specific medical needs, with the device adjusting the dosage in real-time based on feedback from sensors that track the patient's condition.

Given these ongoing challenges in respiratory treatment and emergency drug administration, there is a clear need for innovative systems that address the limitations of traditional inhalation devices. An ideal system would be modular, portable, and easy to use, allowing for precise control over medication dosage while being flexible enough to deliver a wide range of pharmaceutical formulations. Such a system would also need to integrate modern technological advancements, such as sensors for real-time monitoring and feedback, to ensure the highest level of patient safety and compliance.

Despite the advancements in nebulization and inhalation technologies, there is still a significant demand for improved systems that address the practical, technological, and medical challenges present in current devices. As a result, there exists a need for of a more versatile, user-friendly, and technologically advanced formulation delivery system, providing better treatment outcomes for patients suffering from respiratory diseases and ensuring rapid, effective responses in emergency medical situations.

### SUMMARY

Methods and systems for delivering formulations to users using modular device having removable cartridge are disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a formulation delivery system is disclosed. The system comprises system comprises a removable cartridge that includes an atomizer and a wick assembly, ensuring consistent and controlled delivery of medication. The wick, made of an absorbent material such as cotton or ceramic, is configured to absorb a fluid formulation and convey it to the atomizer, where it is converted into an aerosol for inhalation by the user.

A key feature of the invention is the removable cartridge that allows for easy replacement or refilling, making the system adaptable for use with a wide range of medications and formulations. The cartridge is designed with a channel that extends from an outer surface to the wick, enabling the formulation to travel through the cartridge and reach the atomizer for nebulization. The electrical contact at the opposite end of the cartridge engages with the power source of the delivery device, activating the atomizer to create a mist for inhalation.

The modular nature of the system enables it to be integrated into various wearable or attachable devices, such as masks, helmets, and other apparatuses, allowing for hands-free operation in diverse settings. The system also includes sensor technology to detect the presence of the cartridge and initiate the delivery process, further enhancing its reliability and user-friendliness. This modular design makes it particularly suited for patients who require multiple medications or frequent treatments, as it simplifies switching between different formulations without the need for multiple devices.

By addressing the limitations of traditional nebulizers and inhalers, the present invention offers a significant improvement in the flexibility, portability, and precision of inhalation-based drug delivery. The system's interchangeable cartridge, combined with its advanced sensing and atomization capabilities, makes it ideal for delivering both emergency and routine medications, ensuring consistent therapeutic outcomes with minimal user intervention.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1 illustrates a removable cartridge of a device for administering a formulation to a user, according to an embodiment;

FIGS. 2A and 2B illustrate another removable cartridge of a device for administering a formulation to a user, according to an embodiment;

FIG. 3A illustrates a modular device for administering a formulation to a user, according to an example embodiment;

FIG. 3B illustrates a removable cartridge and a base unit of a device, and FIG. 3C depicts a removable cartridge and a mouthpiece for administering at least one formulation to a user, according to an example embodiment;

FIGS. 4A and 4B illustrate a locking mechanism of a formulation delivery system in the unlocked position and the locked position, respectively;

FIGS. 5A and 5B illustrate operation of a removable cartridge of a device for administering at least one formulation to a user, according to another example embodiment;

FIG. 6 is a partially exploded view perspective view of a formulation delivery system according to another example embodiment;

FIG. 7 illustrates an at least partially enclosable wearable apparatus having a first attaching structure, according to an example embodiment;

FIG. 8 illustrates an at least partially enclosable wearable apparatus having a first attaching structure, according to another example embodiment;

FIG. 9 illustrates an at least partially enclosable wearable apparatus having a first attaching structure, according to another example embodiment;

FIG. 10 is a diagram illustrating the main electrical components of a formulation delivery system, according to an example embodiment.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions, or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The disclosed embodiments improve upon the problems with the prior art by providing a formulation delivery system improving portability, dosage accuracy, ease of use, and flexibility in administering different medications. The system comprises a removable cartridge, which serves as the main component for holding and delivering the pharmaceutical formulation. The cartridge includes an atomizer and a wick assembly. The wick, made from an absorbent material such as cotton, ceramic, or synthetic fibers, absorbs the formulation and directs it to the atomizer. When activated, the atomizer converts the liquid formulation into an aerosol mist for inhalation. This modular cartridge design allows for easy replacement or refilling, providing significant flexibility in use, particularly for patients who require multiple medications or frequent treatments.

The cartridge features a channel that extends from the outer surface to the wick, ensuring consistent fluid transport to the atomizer. This design minimizes the risk of clogs or interruptions in the medication flow. The absorbent wick is optimized to ensure that the medication is delivered uniformly to the atomizer, allowing for controlled and consistent aerosolization. In some embodiments, the wick is pre-absorbed with the formulation, reducing the likelihood of spills or contamination during cartridge handling. This pre-absorption approach also enhances ease of use, as patients do not need to handle the liquid directly when replacing or refilling the cartridge.

To ensure proper operation, the cartridge is equipped with electrical contacts that interface with the main device's power source. These electrical contacts provide the necessary energy to activate the atomizer, ensuring the formulation is nebulized when the device is in use. The design of the electrical contacts ensures stable and consistent power transmission, even in situations where the atomizer is vibrating during operation. Additionally, the electrical contacts can transmit signals between the cartridge and the control system, enabling real-time monitoring of dosage and device status.

The modular nature of the system allows it to be integrated into a wide range of wearable or attachable devices, making it suitable for various medical and emergency environments. For example, the removable cartridge can be inserted into a wearable mask or helmet, allowing hands-free operation in situations where portability and ease of use are critical. The system's design ensures that the cartridge is securely attached to the wearable device using locking mechanisms and air-tight seals, preventing contamination of the medication, and ensuring consistent delivery during use.

The system also includes sensor technology to further enhance user experience and device reliability. Sensors within the device can detect the presence of the cartridge, ensuring that fluid delivery only occurs when the cartridge is properly inserted. This prevents user errors such as incomplete insertion or misalignment of the cartridge. Sensors may also monitor the fluid levels within the cartridge, providing feedback to the user when the cartridge needs to be replaced or refilled. Additionally, sensors can regulate the activation of the atomizer, ensuring that the medication is aerosolized at the appropriate time and dosage, based on predefined conditions.

The integration of modern sensing and feedback technologies allows the system to provide real-time information to the user through visual or audio indicators. For example, the system may include a graphical display or indicator lights that notify the user when the cartridge is properly engaged or when an error condition is detected. Audio feedback, such as beeps or voice prompts, can further guide the user in operating the device, making it accessible for a wide range of patients, including those with limited technical expertise.

The removable cartridge can hold a variety of pharmaceutical formulations, including solutions, suspensions, and emulsions, depending on the therapeutic need. Solutions are often used for medications that require rapid and uniform delivery, while suspensions are ideal for drugs that require controlled release over time. The use of emulsions allows for more complex formulations, where two immiscible liquids are combined to enhance drug absorption or provide buffered release. Each formulation is delivered consistently through the wick and atomizer, ensuring effective treatment across different medical conditions.

In one embodiment, the system is designed to accommodate pre-filled cartridges that are specifically tailored for certain medications or therapeutic applications. Pre-filled cartridges ensure that the exact dosage is provided, reducing the risk of user error, and making the system ideal for emergency use. For example, a cartridge pre-filled with naloxone can be used for rapid administration in cases of opioid overdose, ensuring that the medication is delivered effectively and promptly. Other cartridges could be filled with yohimbine to reverse the effects of xylazine, a veterinary sedative increasingly found in contaminated street drugs.

The wearable and attachable designs also enhance the system's versatility, making it suitable for use in critical environments such as hazardous material suits or space suits. These designs ensure that the patient can receive medication without removing their protective gear, a feature particularly valuable in high-risk or emergency settings. The valve systems integrated into these devices allow for controlled airflow and prevent environmental contamination, maintaining the integrity of the medication throughout its use.

By utilizing modular components, the system is designed to be adaptable to a wide range of applications while maintaining user-friendliness and precision. This adaptability ensures that patients with chronic conditions, as well as those in emergency scenarios, can receive the medication they need in a timely and effective manner. Furthermore, the control module within the device allows for automated dosage regulation, minimizing the need for manual intervention and providing peace of mind that the correct amount of medication is being delivered at the right time.

In addition to its practical benefits, the system's modular design reduces overall costs for users. By enabling multiple medications to be delivered using a single device, patients no longer need to invest in separate nebulizers or inhalers for each medication. This reduces the financial burden on patients and streamlines the medication management process. Moreover, the pre-filled, easy-to-use cartridges reduce the need for frequent medical visits for device maintenance or refills.

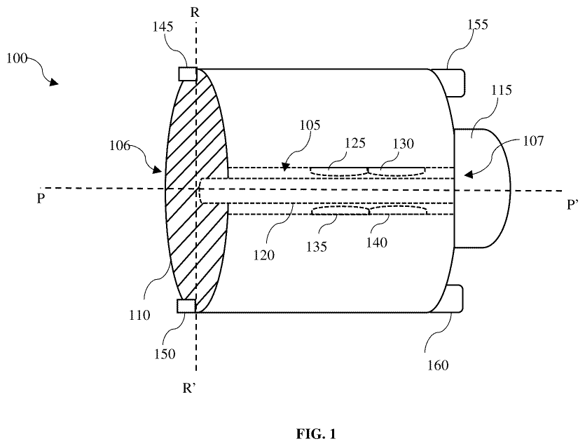

Referring now to the Figures, FIG. 1 illustrates the removable cartridge designed for use in a formulation delivery system. This figure showcases the removable cartridge 100, which includes an elongated channel 105 extending from a first end portion 106 to a second end portion 107, an atomizer 110 disposed at the first end portion 106, a wick assembly with a removable cap 115 and a wick 120, and guides 125, 130, 135, 140. The wick 120 comprises an absorbent material for absorbing a fluid, wherein one end of the wick 120 abuts a portion of the atomizer 110 and is configured to covey the fluid to the atomizer. In some embodiments, the absorbent material is cotton. In other embodiments, the absorbent material may be silica, ceramic, stainless steel mesh, rayon, hemp, or any other suitable material. The absorbent material is configured to store a fluid such as a medicinal formulation. The absorbent material is configured to release the fluid formulation upon activation of the formula delivery system by the user. In the context of a formulation delivery system, the removable cartridge serves as the reservoir for a medicated formulation. The interchangeable nature of the cartridge allows for ease of refilling or replacing the medication, thus offering flexibility and convenience to the user. This feature is particularly beneficial for patients requiring multiple medications or dosages, as it simplifies the process of switching between different medication types without the need for multiple devices. The removable cartridge, which is cylindrical in this depiction, could alternatively be designed in various shapes such as rectangular, oval, or customized forms to fit specific devices or user preferences. The shape of the cartridge is often dictated by ergonomic considerations, the volume of medication it needs to hold, and how it will interface with the device. For instance, a flat, disc-shaped cartridge might be employed to create a more compact device, while a larger cylindrical shape might be used to contain a greater volume of medication for devices intended for multiple doses or extended use.

Within the cartridge, the elongated channel 105 runs the length of the cartridge from the first end portion 106 to the second end portion 107. This channel is sized to accommodate the dimensions of the wick, ensuring that the wick can be saturated with medication while maintaining the necessary capillary action to draw the medication towards the atomizer. In an embodiment, the elongated channel extends from a portion of an outer surface of the removable cartridge to wick, wherein, the outer surface may be located on the first end portion 106 or the second end portion 107 or both. The wick assembly, integral to the function of the device, features the removable cap 115, and the wick 120, which are designed as a singular unit. The design is such that removing the cap from the cartridge also withdraws the wick from the channel, and reinserting the cap positions the wick back into the channel. This setup is strategically devised so that the cap remains external to the channel while the wick resides within, fully inside the channel. In various embodiments, the channel may contain the formulation, and the wick may be immersed in the formulation. Other embodiments, where the channel does not contain the formulation and houses the wick is also covered within the scope of the invention. The wick within a medication device is a crucial component that absorbs the medication and, upon activation, directs it toward the atomizer for nebulization. The wick's material is an absorbent material and is selected based on its absorbency and compatibility with the formulation, often being made from cotton, synthetic fibers, or ceramic materials. Its utility lies in its ability to provide a consistent and controlled delivery of medication, which is particularly important for ensuring accurate dosages and effective treatment.

Guides 125, 130, 135, and 140, also referred to as alignment ribs, are affixed to the interior wall of the channel to ensure the correct placement and stabilization of the wick within the cartridge. In the embodiment, the wick is affixed within the removable cartridge. These alignment ribs can vary in shape, such as straight, curved, or angular and can be made from a range of materials that are chosen for their durability and compatibility with the medication. For instance, plastic ribs might be used for their resilience and moldability, while metal ribs could be selected for their strength and rigidity. The size of these ribs is also variable, designed proportionally to fit the internal dimensions of the channel and the size of the wick. The atomizer is strategically located at one end of the cartridge, opposite the end where the removable cap is placed. In this configuration, the wick is oriented transversely or substantially perpendicular to a plane of the atomizer, allowing for an effective transfer of medication. The perpendicular or transverse arrangement of the wick's longitudinal axis, PP′, to the atomizer's longitudinal axis, RR″, ensures an efficient pathway for the formulation to reach the atomizer. This orientation enables one end of the wick to maintain direct contact with the atomizer, allowing the formulation to be conveyed efficiently for nebulization. The relative position of the wick being substantially perpendicular to the atomizer allows the entire cross-sectional surface of the wick to abut directly against the atomizer. This specific configuration ensures that the maximum surface area of the wick is in contact with the atomizer, facilitating a complete and consistent transfer of the liquid formulation from the wick's reservoir to the atomizer. Further, the channel extends from a portion of an outer surface of the removable cartridge to the wick and the capsule is disposed within the channel such that the capsule is arranged transversely to a longitudinal axis of the wick. Additionally, the cartridge is fitted with two attachments 145 and 150 at the first end portion 106, and is equipped with electrical contacts, 155 and 160, at the second end portion 107, enabling it to interface with the power source of a medication delivery device, ensuring that the atomizer receives the necessary energy to aerosolize the medication for inhalation. This design provides precision in medical device manufacturing, where every component must work synergistically to deliver safe and effective treatment to patients.

The electrical contacts are designed to interface with corresponding contacts on a subsequent section. Regarding the first section, it integrates with the second section and the second section further integrates with the third section. These contacts are strategically placed to ensure a reliable electrical connection upon the assembly of the two sections. The primary function of these contacts is to facilitate the transfer of power and control signals between the sections. For instance, when the cartridge, containing the medicated fluid, is attached to the first section, the electrical contacts activate the atomizer within the first section, initiating the process of converting the fluid into an aerosol.

The electrical contacts in the receiving section may include an electrical mating portion. This mating portion is designed to ensure a consistent and uninterrupted electrical connection between the receiving section and the interchangeable cartridge, even when the atomizer is in operation and vibrating. The vibration of the atomizer, necessary for converting the medicated fluid into an aerosol, presents a potential challenge for maintaining a stable electrical connection. To address this, the electrical mating portion is engineered to accommodate movement without losing contact. This could be achieved through the use of spring-loaded contacts, flexible conductive materials, or a design that allows for a certain degree of movement while still maintaining an electrical connection.

The electrical mating portion is designed to be robust and to provide a secure connection that can withstand the mechanical stress caused by the atomizer's vibration. This design ensures that there is no disruption in the power supply or control signals between the sections of the device. It is essential for the reliable function of the atomizer and, consequently, for the effective delivery of the medication. Additionally, the electrical mating portion may be integrated in a way that aligns effortlessly with the corresponding contacts on the cartridge. This ease of alignment is important for ensuring that the device is user-friendly and that the process of changing cartridges is straightforward and error-proof.

Each section may feature electrical contacts designed for connection with another section. These contacts are crucial for the transmission of power and communication signals between the sections. The electrical contacts are strategically positioned to align with corresponding contacts in an adjacent section. This alignment ensures a secure and efficient electrical connection when the sections are assembled. The contacts are typically made of conductive materials known for their durability and resistance to corrosion, such as gold or silver alloys, to ensure a reliable connection over the lifespan of the device. The design of these contacts considers the need for a stable connection that can withstand regular use. This includes considerations for easy alignment and connection, ensuring that when the second and third sections are joined, the electrical contacts meet with minimal effort from the user. This user-friendly design is essential for the regular replacement or refilling of the cartridge in the second section.

In some embodiments, the electrical contacts may include features such as spring-loaded pins or pressure contacts. These features ensure a consistent electrical connection even when there is slight movement or misalignment between the sections. They provide the necessary flexibility while maintaining a strong electrical contact, crucial for the uninterrupted operation of the atomizer and other electronic components in the device.

Furthermore, the electrical contacts are designed to facilitate not just power transmission from the third section's power source to the atomizer in the first section via the second section but also to allow communication signals to be sent and received. This includes signals related to the control of the atomization process, feedback from sensors in the second section, and information display on the user interface.

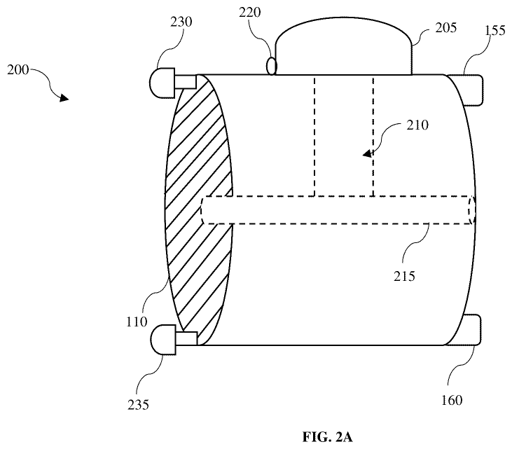

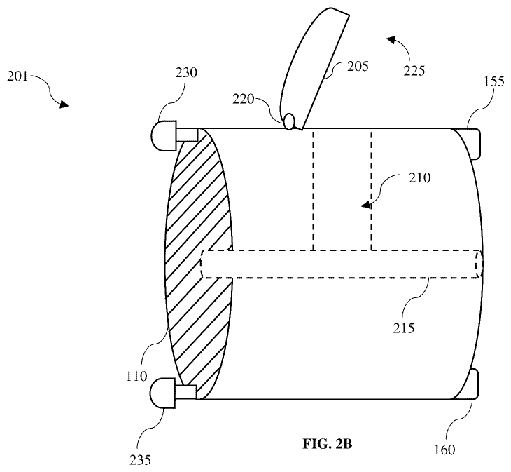

FIG. 2A illustrates an exemplary removable cartridge 200 having a distinct variation from the embodiment outlined in FIG. 1. The embodiment of FIG. 2A has the positioning of the removable cap 205, which is situated on the side of the atomizer of the removable cartridge, rather than on an opposite end of the atomizer. The removable cartridge contains a channel 210, which is an elongated passage extending from a section of the cartridge's outer wall near the cap to the wick 215. This channel is critical as it forms the path through which the medication travels from its storage area within the cartridge to the wick, ready for atomization. In this embodiment, the channel 210 is substantially perpendicular or transversely to a plane of the wick 215, a design that suggests intentionality in how the medicated formulation is delivered to the wick. The wick is securely attached within the body of the cartridge, fixed at one inner wall, and reaching out to the atomizer at the opposite end. This attachment can be achieved through a variety of methods. For example, the wick may be embedded into the cartridge during the manufacturing process, mechanically fastened with small clips or tabs, or held in place with non-reactive, medical-grade adhesives. The chosen method typically balances the need for a firm hold to prevent movement during use and the convenience of replacement if necessary.

The perpendicular orientation of the wick to the atomizer plays a pivotal role in the uniform distribution of the medicated formulation. When the wick is attached centrally to the atomizer, it allows for an even spread of the liquid across the atomizing component, contributing to a consistent aerosol output. The advantages of this design include more efficient medication delivery, leading to potentially better therapeutic outcomes, minimized wastage of medication due to uneven spreading, and the prevention of hotspots that could lead to inconsistent atomization. The cap's attachment mechanism is represented in the drawing by a hinged connection, which allows the cap to swing open. FIG. 2B illustrates an exemplary removable cartridge 201 wherein the removable cap is in the open position. The hinge 220, enables a controlled opening of the cap, allowing the user to refill the channel 210 with medication as needed. The figure indicates the cap's pivotal movement along the direction marked as 225. This hinge system ensures that when the cap is in the closed position, the channel 210 is fully covered, maintaining the sterility of the wick and the medication. In an open position, the cap exposes the channel, providing access for medication refills for absorption by the wick. Other methods of cap attachment might include a slide-and-lock mechanism, where the cap slides into place before locking, or a magnetic system that allows for a secure closure without physical clips or latches.

In this embodiment, the insert tabs, such as 230 and 235, are positioned on one end of the cartridge and form a second attaching structure. These tabs, with their rounded edges, are designed for effortless insertion into the corresponding part of an attachment such as a first attaching structure. Pushing the cartridge into the first attaching structure until the tabs click into place ensures the cartridge is securely attached, providing the user with a clear indication that the device is ready for use. The design of these tabs is critical not only for a secure fit but also for ensuring ease of cartridge replacement by the user, contributing to the overall practicality and maintenance of the medication delivery device.

In the disclosed embodiments, the formulation is not directly contained in the removable cartridge but instead the wick is pre-absorbed with the formulation that is used. This design significantly reduces the likelihood of leaks and spills, as the liquid is securely held within the wick's fibers, which can enhance the device's reliability and user confidence during transport and use. The pre-absorption method ensures that the formulation is evenly distributed across the wick, promoting consistent vapor production and efficient utilization of the substance without the need for frequent refills. This setup also simplifies the cartridge replacement process, as users can swap out the wick assembly without handling the liquid directly, making the process cleaner and more convenient. Additionally, this approach can improve the longevity of the device by minimizing the exposure of liquid to sensitive components such as the atomizer and electrical contacts, thereby preserving their function and reducing maintenance needs. Overall, the use of a pre-absorbed wick in a removable cartridge enhances the practicality, efficiency, and user-friendliness of the device.

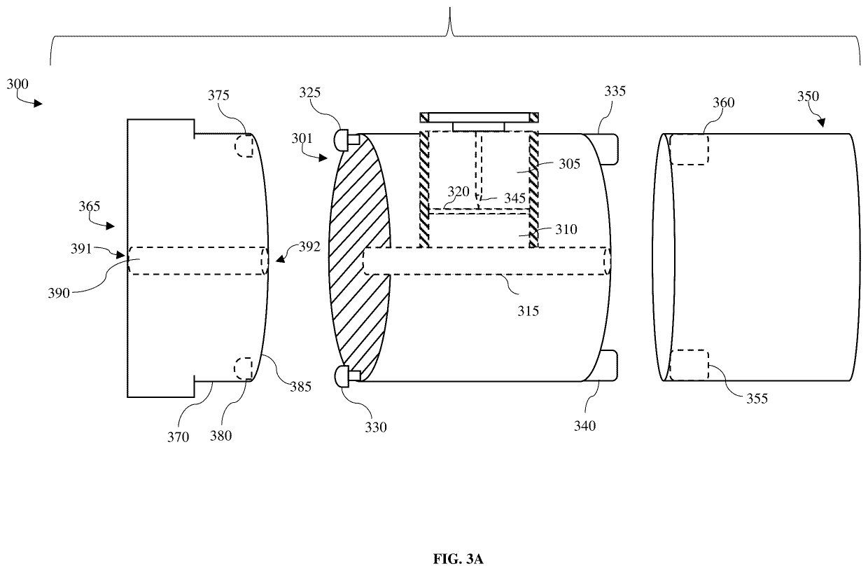

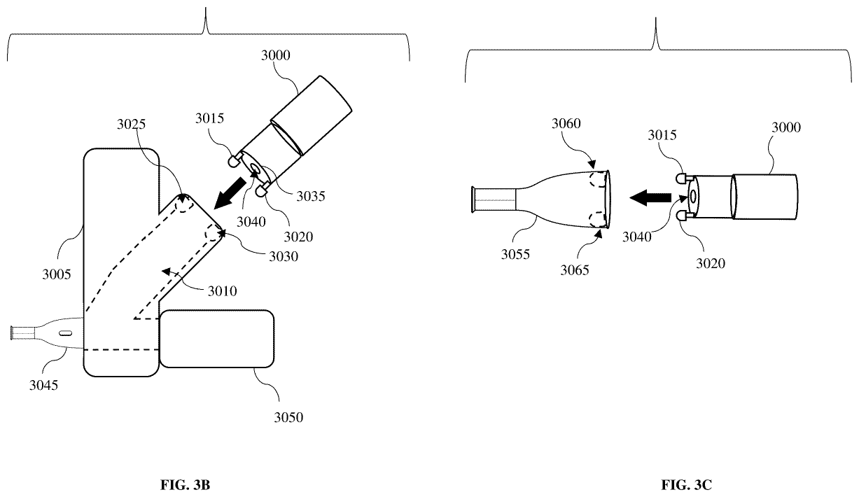

FIG. 3A illustrates an embodiment of a device 300 having a removable cartridge 301, an electronic device 350, and a first attaching structure 365. The removable cartridge has a capsule defining more than one chamber, namely, a first chamber 305 for holding the formulation therein and a second chamber 310 that is in direct contact with a portion of the wick 315 for absorbing the formulation by the wick once the capsule is engaged and/or activated. Said chambers are initially separate until the capsule is engaged as to breach a divider or a sealing member 320 between the chambers enabling the first and second chamber to be in fluid communication with one another. The capsule may include a rupturing element 345, such as a needle, which can puncture the sealing member 320 between the first chamber and the second chamber. The removable cartridge has insert tabs 325, 330 on one end, and a first pair of electrical contacts 335, 340 on an opposite end. The insert tabs are configured to engage with a first attaching structure 365, via a receiving port 375, 380 and an air tight seal 385.

The first attaching structure may be directly or indirectly attached to a wearable device or article of clothing, such as, a mask, suit, or mouth piece. The first attaching structure may be integrally formed as part of a wearable device, such as a mask, suit, or mouthpiece. This means the attaching structure is built directly into the material or design of the wearable, offering a seamless connection for the removable cartridge. For respiratory masks used in medical settings, such as CPAP devices or inhalation therapy masks, the first attaching structure can be molded directly into the mask's body. This allows for the quick and efficient insertion of a medication cartridge, ensuring a secure, air-tight fit and delivering the medication directly into the user's respiratory pathway. In cases like hazardous materials (hazmat) suits, space suits, or other protective gear, the attaching structure could be integrated into the fabric or hard shell of the suit. This enables the wearer to receive medication while remaining fully protected from external contaminants. The structure could be placed near the chest, neck, or helmet, allowing for quick access without compromising the suit's protective functions. In a mouthpiece, the attaching structure can be integral to the design of the inhalation device, such as a nebulizer or inhaler mouthpiece. This direct integration allows for the removable cartridge to be easily inserted, with a snap-fit or similar mechanism ensuring that the cartridge remains securely in place during inhalation. In other embodiments, the first attaching structure may be indirectly attached to the wearable device or clothing. This allows for modularity and flexibility, where the attaching structure can be fixed to the wearable device but easily removed or repositioned as needed. Different fastening mechanisms may be used for this purpose, including, clips or clamps, straps and bands, or magnetic fasteners. The attaching structure could be attached to a wearable device using clips or clamps, which would allow the user to reposition the cartridge holder on various parts of the device, depending on user preference or medical need. Elastic straps or adjustable bands would allow the first attaching structure to be fastened securely around various wearable articles, such as a suit sleeve, a belt, or a helmet. The first attaching structure may be secured with magnetic fasteners, allowing it to be quickly attached or detached from the wearable article.

The first attaching structure further comprises an elongated channel 390 extending from a first end portion 391 to a second end portion 392. When the insert tabs are fully received by the receiving ports, the locking mechanism is engaged, and the formulation delivery system is in the locked configuration. In the locked configuration, the air tight seal 385 is designed to maintain the integrity of the medication delivery system by preventing contamination from external environments and ensuring a secure, leak-free connection between the removable cartridge and the main device or wearable apparatus. The air tight seal is located at the interface between the removable cartridge and the first receiving port of the device. It is typically made from a flexible, durable material such as silicone, rubber, or another medical-grade polymer that can create a firm seal when the cartridge is inserted into the device. The flexibility of the material allows it to conform closely to the surfaces of both the cartridge and the receiving port, ensuring that no air or external particles can enter the system once the cartridge is locked into place. The air-tight seal is activated upon the locking mechanism engaging the removable cartridge within the device. When the cartridge is fully inserted, the seal compresses to create a tight, secure fit, allowing the system to function optimally in both medical environments and more extreme settings, such as emergency situations or when integrated into wearable devices like space suits, hazmat suits, or masks. The electrical contacts are configured to engage in removable electrical communication with an electronic device 350, such as, a battery, via a second pair of electrical contacts 355, 360. The electronic device 350 houses the system's power source, which can be a rechargeable battery or an external power source. It provides the necessary electrical energy to the atomizer within the removable cartridge, enabling the atomization process. The device regulates power flow to ensure consistent and controlled operation, activating the atomizer only when required. This helps to optimize power usage, prolong battery life, and ensure that the device is always ready for use when needed. The electronic device 350 is equipped with electrical contacts 355, 360 that interface with the electrical contacts 335, 340 of the removable cartridge. These contacts are responsible for transferring power from the electronic device to the atomizer within the cartridge. When the cartridge is inserted, the electronic device and the removable cartridge are in removable electronic communication, the electrical contacts establish a reliable connection, allowing the atomizer to receive the necessary power for generating aerosol from the liquid formulation.

FIG. 3B depicts an embodiment wherein the removable cartridge 3000 is configured to integrate with a first attaching structure 3005. The first attaching structure is sometimes referred to as a base unit. The first attaching structure is constructed with a receiving chamber containing a channel 3010, deliberately designed to receive the cartridge. A precise snap-fitting mechanism is embodied by connectors or insert tabs 3015, 3020 on the cartridge that snugly fit into receiving ports 3025, 3030 first attaching structure, ensuring a secured connection. In an embodiment, as illustrated in FIGS. 3B and 3C, the atomizer 3035 is designed with a single hole 3040 for nebulization at the center instead of a mesh across the entire surface. Additionally, the atomizer features a flat area around the hole where the connectors or insert tabs 3015, 3020 are located. The atomizer 3035, located at one end of the removable cartridge 3000, is a focal component of the device, facilitating its primary function. The removable cartridge 3000 and first attaching structure 3005 are constructed from a high-grade polymer, selected for its durability, chemical resistance, and suitability for precision molding. The material ensures the integrity of the device even after repeated use and exposure to the substances to be atomized. Both components are manufactured using an injection molding technique, which allows for the production of parts with complex geometries and tight tolerances, essential for the snap-fit functionality and proper alignment of the atomizer. It is noted that the disclosed embodiment illustrates an atomizer with a single hole at the center, however, other types of atomizers, which features a mesh across the entire surface as shown in other embodiments, and atomizer having multiple holes are also covered within the spirit and scope of the invention.

The primary attachment mechanism of the invention is characterized by a snap-fit design, allowing for a secure yet user-friendly interface between the removable cartridge 3000 and first attaching structure 3005. Specifically, the insert tabs 3015, 3020 on the cartridge are engineered to slide into and lock within the receiving ports 3025, 3030. While this embodiment showcases a mechanical snap-fit approach, alternative embodiments may incorporate different attachment strategies such as magnetic coupling, where magnets embedded in the components create a reversible and clean connection. Other possible attachment means include twist-and-lock mechanisms, where components are joined by aligning and twisting into a locked position, latch-based systems, which employ a movable latch or catch to secure components together, and protrusion tabs. These varied attachment options provide flexibility in design and user interaction, catering to different applications and preferences. The operation of the atomizer 3035 employs a piezoelectric element that vibrates at ultrasonic frequencies when energized, creating a fine mist from the liquid contained within the cartridge. This efficient atomization process is optimized for minimal waste and maximum dispersion of the atomized product. The operation and activation of the atomizer in the described device are managed by electronics housed within the removable cartridge, ensuring a streamlined and efficient user experience. This electronic assembly includes a printed circuit board (PCB) and a battery, which together form the control center for the atomizer. The PCB is programmed to regulate power delivery from the battery to the atomizer based on user inputs and pre-set conditions. This setup allows for precise control over the atomization process, enabling features such as adjustable vapor output and temperature settings, which can be tailored to individual preferences for an optimal vaping experience. The inclusion of the battery within the cartridge not only centralizes the power source but also simplifies the design, allowing for compactness and portability. By integrating these components, the cartridge becomes a self-contained module that can be easily replaced or upgraded, enhancing the device's convenience and functionality. This system not only increases the ease of maintenance and the reliability of the device but also provides users with a high degree of control over their vaping experience.

The first attaching structure displayed in the illustration is equipped with multiple openings, each serving as a potential point of attachment for various additional parts, augmenting the device's functionality. One of these openings is designed to accommodate attachments such as a mask or a mouthpiece 3045, which directs medicated aerosol to the patient's nose and mouth. Additionally, a resilient bladder or an ambu-bag 3050 can be attached to other openings as shown in FIG. 3B. This setup may serve either as a manual means for generating aerosol or as a reservoir for storing medication. These components can be designed for a snap-fit, twist-lock, or magnetic attachment, ensuring a secure and user-friendly interface. The configuration described is particularly useful in clinical settings such as hospitals. It facilitates the direct administration of medicated aerosols to patients, either through manual operation or automated delivery systems. The versatility of the first attaching structure's design allows it to be customized according to the user's needs, facilitating a range of functions from direct inhalation therapy to assisted delivery mechanisms, thereby enhancing the device's applicability for various respiratory treatments.

FIGS. 3B and 3C depict the removable cartridge 3000 being interchangeable with the first attaching structure 3005 and a mouthpiece 3055. In FIG. 3B, the removable cartridge attaches to the first attaching structure, and in FIG. 3C, the removable cartridge connects to the mouthpiece 3055 using the insert tabs 3015, 3020. These tabs are inserted into corresponding receiving ports 3060, 3065 of the attachment on the inner wall of the mouthpiece. The insert tabs fit snugly into the receiving ports of the mouthpiece 3055. Both receiving ports 3060, 3065 are strategically positioned on the inner wall of the mouthpiece to ensure a secure coupling. This design facilitates a robust connection and allows for easy assembly and disassembly. The utilization of same tabs for modular attachment to various components, such as the first attaching structure and the mouthpiece offers significant advantages in terms of device modularity. This uniformity allows the removable cartridge to be used across different applications and requirements, enhancing the versatility of the device. For instance, as depicted in FIG. 3C, this modularity contributes to a compact structure that is easily portable, enabling users to carry the device discreetly in their pockets and transport it to various locations without hassle. Such a design not only meets diverse user needs but also maximizes the functional adaptability of the device, catering to various usage scenarios with ease. The embodiment shown in FIG. 3C features a specific design of the mouthpiece, it is noted that the scope of the invention includes various other shapes and designs of the mouthpiece as detailed in other embodiments. This inclusivity ensures that the invention can be adapted to different use cases and preferences, making the device applicable in various settings.

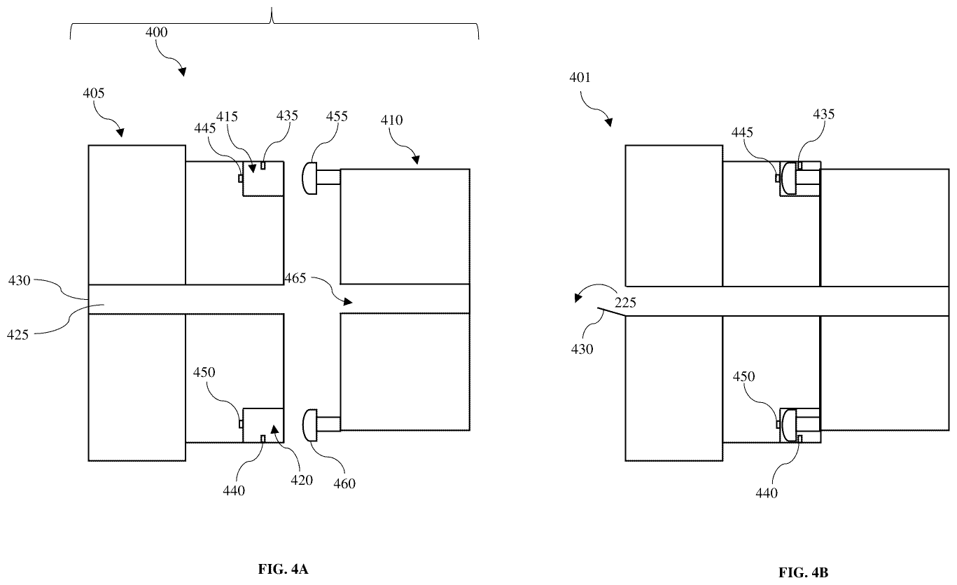

FIGS. 4A and 4B are cross-sectional views of a first attaching structure 405 and a removable cartridge 410 in the unlocked position 400 and locked position 401, respectively. The first attaching structure comprises a receiving port 415, 420, elongated channel 425, and a valve system 430 comprising of a check valve and air lock. The receiving port further comprises a locking mechanism 435, 440 and sensor 445, 450. The removable cartridge comprises insert tabs 455, 460 and an elongated channel 465. In the unlocked position, the first attaching structure and the removable cartridge are not engaged with each other and the valve system 430 is in a closed position, preventing the flow of air or fluid.

The valve system, sometimes referred to as the valve, ensures that the system remains sealed, and that medication delivery is regulated properly when the removable cartridge is engaged or disengaged. The valve system's design is integral to maintaining environmental isolation, preventing contamination, and allowing for controlled airflow or medication delivery. The valve system contributes to the air-tight seal of the system. When the cartridge is not engaged, the valve remains closed, ensuring that no external air, moisture, or contaminants can enter the system. This preserves the sterility of the medication within the cartridge. Once the removable cartridge is fully inserted into the first attaching structure, the valve opens, creating a controlled and sterile path for the medication to be aerosolized and delivered to the user. The valve may be pressure-activated, meaning it opens when the cartridge is fully inserted, and pressure is applied to the locking mechanism. This ensures that the valve only opens when the system is correctly engaged and ready for use. In some embodiments, the valve could be designed as a one-way valve. This means that air or medication can flow in only one direction-outward toward the patient-preventing any backflow of air or contaminants into the cartridge or device. A check valve may also be implemented as part of this design, ensuring that once the medication is aerosolized and delivered, no external air can re-enter the system through the valve, preserving the quality and dosage of the medication. The valve is intricately linked to the locking mechanism. In the unlocked (disengaged) position, the valve remains sealed, blocking airflow or fluid exchange. In the locked (engaged) position, the valve opens automatically once the locking mechanism confirms that the cartridge is securely in place. This synchronization ensures the system only becomes operational when it is properly assembled and ready for use.

The sensors 445, 450 sensors are designed to optimize the delivery of medication, prevent user errors, and ensure the correct operation of the formulation delivery system. The sensors are responsible for detecting whether the removable cartridge is properly inserted into the receiving port of the device and in the locked position 401. These sensors ensure that the system only activates when the cartridge is correctly engaged, preventing accidental or improper operation. The sensors can be selected from a group consisting of magnetic sensors, optical sensors, or pressure sensors. In some embodiments the sensor is a magnetic sensor, the magnetic sensor detects changes in the magnetic field when the cartridge, which may contain a magnetic element or trigger, is inserted. In some embodiments the sensor is an optical sensor, the optical sensor uses a light beam or infrared technology to detect the cartridge's presence by measuring whether the light is blocked or reflected when the cartridge is inserted. In some embodiments the sensor is a pressure sensor, the pressure sensor detects the physical pressure applied when the cartridge is locked into place, confirming that the cartridge is fully engaged. When the sensor detects the correct insertion of the cartridge, it sends a signal to the control system to activate the atomizer and initiate a fluid delivery. This prevents the system from attempting to nebulize medication when the cartridge is not present or properly aligned, avoiding potential errors or malfunction. Upon proper insertion, the sensor may also send a signal to the valve system 430 causing it to transition to an open position, as illustrated in FIG. 4B.

To engage the locked position 401, the removable cartridge is directly inserted into the receiving port by way of the insert tabs. When the insert tabs are fully received by the receiving port, the locking mechanism will engage, securing the removable cartridge to the first attaching structure in a locked position. This ensures that the cartridge cannot be accidentally removed or displaced during use. The locking mechanism may use clips, latches, magnets, twist-and-release, or spring-loaded elements to hold the cartridge securely, creating a stable connection between the cartridge and the device. In the locked position, there is an air tight seal between the removable cartridge and first attaching structure.

In the locked position, the elongated channel 425 of the first attaching structure is in direct alignment with the elongated channel 465 of the removable cartridge and the valve system 430 is in an open position. In the locked position, the fluid pathway between the removable cartridge and the first attaching structure is established and the atomizer is positioned correctly for efficient aerosolization of the medication, allowing medication to flow properly. In the locked position, the proper alignment of the elongated channels ensures that the fluid pathway from the cartridge to the atomizer is fully open, allowing the liquid medication to be conveyed to the atomizer without obstruction.

To disengage the cartridge from the locking mechanism and remove it from the receiving port, there are several possible methods that could be employed, depending on the specific design of the locking mechanism. These methods ensure a secure yet user-friendly process for detaching the cartridge. A push-button mechanism could be included on the external surface of the first attaching structure or the device itself. The user would press this button to disengage the locking mechanism, releasing the cartridge from the receiving port. When the button is pressed, it could trigger a spring-loaded or latch-based system inside the locking mechanism, causing the internal locking clips to retract, thereby releasing the cartridge. A twist-and-release mechanism could be implemented, where the user twists the removable cartridge a certain number of degrees (e.g., 90 or 180 degrees) to disengage the locking mechanism from the corresponding slots in the receiving port. The twisting motion could cause the locking mechanism 435, 440 to move out of alignment with the insert tabs 455, 460 on the cartridge, thereby disengaging the cartridge from the receiving port. Once twisted to the unlocked position, the cartridge can be pulled out. A sliding mechanism could be used, where the user slides a small latch or lever along the body of the device or the cartridge to unlock the cartridge from the locking mechanism. If the system employs magnetic locking elements, the user could apply a counter-magnetic force or simply move a magnetically equipped release tool near the locking area to disengage the mechanism. A depress-and-release mechanism, wherein, the user depresses a part of the cartridge itself or an external component to release it from the locked position. The action of pressing down on a specific area could cause internal latches or clips to retract, disengaging the cartridge from the receiving port. After the release, the cartridge can be easily pulled out of the system.

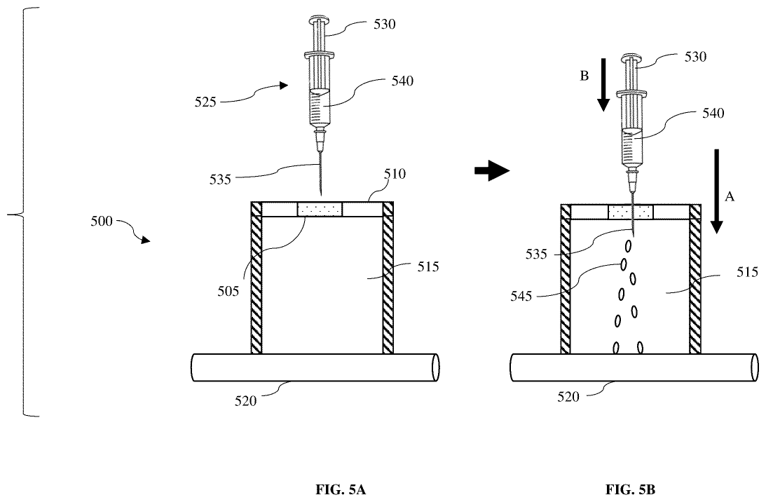

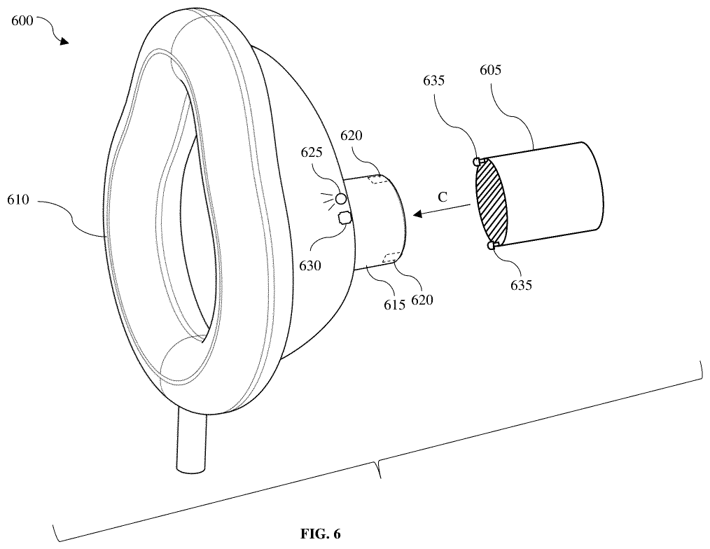

FIGS. 5A and 5B illustrate an embodiment where a capsule 500 of a device as exemplified in FIG. 3A has a rubber section as a covering member 505 covering an open portion on a top side 510 of the capsule. The capsule includes a chamber 515 for receiving a formulation and a rubber section as the seal or covering member 505 covering the open portion of the chamber of the capsule. The rubber section allows for a formulation to be inserted into the capsule. The capsule further includes a wick 520 proximate to a second or bottom side of the capsule. One end of the wick, as shown in FIG. 3A abuts the atomizer. In operation, a user of the capsule may add formulation by inserting a syringe through the rubber section and using the syringe to dispense the formulation into the chamber 515. As shown in FIGS. 5A and 5B, the syringe 525 having a plunger 530, and a needle 535 contains the formulation 540 and the syringe is inserted through the covering member 505 into the chamber 515. The syringe is inserted along a direction A such that a portion of the needle 535 is positioned inside the chamber 515, shown in FIG. 5B. Once inserted, the plunger 530 of the syringe is drawn with a downward force applied in the direction B, as shown in FIG. 5B. The formulation is released by the needle in form of droplets 545 and received by the portion of the wick 520. The formulation is absorbed by the wick and conveyed to the atomizer. The capsule chamber is above the wick 520 and abuts the wick 520 such that gravity allows the formulation to be received and absorbed by the wick. Gravity forces the formulation down such that the formulation presses down against the wick. In an embodiment, there may be a sensor implemented in the capsule. For example, the sensor may be a float sensor that measures the level of liquid in the capsule chamber. The float sensor is a continuous level sensor featuring a magnetic float that rises and falls as liquid levels change. The movement of the magnetic float creates a magnetic field that actuates a hermetically sealed reed switch located in the stem of the level sensor, triggering the switch to open or close. Other types of sensors configured to detect the amount of liquid in the capsule chamber may be used and are within the spirit and scope of the present invention. Additionally, the maximum amount of formulation or time may be adjusted depending on the patient, medication, and variety of other factors.

In an embodiment, the covering member 505 is a removeable covering, such as, but not limited to, a cap or seal, in attachment with the top side 510 of the capsule to preserve the formulation and/or prevent the formulation from leaking. The removeable covering allows users of the system to store capsules for emergency use or long-term use, depending on the type of removeable covering. In some embodiments, the capsule may be color-coded for emergency medication or may include labels that identify the medication within the capsule. The capsule may also include a locking element that prevents the capsule from atomizing the medication unless an access code is provided.

In the embodiments described, a variety of liquid formulations can be utilized depending on the specific medical or therapeutic application intended. These formulations may include, but are not limited to, solutions, suspensions, and emulsions. Solutions are homogeneous mixtures where the medication is completely dissolved in a solvent, typically water or an organic solvent like ethanol, which ensures rapid and uniform delivery upon administration. Suspensions are heterogeneous mixtures where the medication particles are dispersed throughout the solvent but are not dissolved; this form is useful for substances that are insoluble or unstable in a solvent, providing a controlled release as the particles slowly dissolve over time. Emulsions, which are mixtures of two immiscible liquids where one is dispersed in the other as fine droplets, often include an oil phase and a water phase, and are particularly beneficial for drugs that require a buffered release or enhanced absorption. Each of these formulations can be tailored with additives such as stabilizers, buffers, and preservatives to enhance the stability, efficacy, and shelf life of the medication, thereby providing significant improvements over prior art by optimizing drug delivery and patient compliance.

FIG. 6 is an embodiment of a formulation delivery system 600 comprising an exemplary removable cartridge 605 and an at least partially enclosable wearable apparatus 610. The at least partially enclosable wearable apparatus, sometimes referred to as a wearable apparatus, is at least partially enclosable and comprises a first attaching structure 615, wherein, the first attaching structure further comprises a receiving port 620, indicator light 625, and control module 630. The at least partially enclosable wearable apparatus as illustrated in FIG. 6 is a face mask similar, such as, an oxygen mask, however, it is understood that other types of face masks may be used instead. The oxygen mask comprises a first attaching structure 615 that is configured to receive the removable cartridge, The removable cartridge comprises insert tabs 635 configured to be received by the receiving port 620, as exemplified in FIGS. 4A and 4B. The removable cartridge is inserted into the receiving port in the C direction. When the removable cartridge is fully inserted into the first attaching structure the insert tabs 635 are engaged with the receiving port 620 and in a locked position, as exemplified in FIG. 4B. The indicator light 625, sometimes referred to as a perceivable indicator, provides visual feedback to the user about the status of the system. This perceivable indicator could signal various conditions, such as, whether the cartridge has been properly inserted and locked into place, whether the system is powered on and ready for operation, and alerts related to low medication levels, malfunction, or other operational issues. In some embodiments, the indicator light is a perceivable indicator that emits a perceivable signal indicating proper engagement of the removable cartridge to the first attaching structure. In further embodiments, the perceivable indicator emits a second perceivable signal indicating an error condition, such as, improper coupling of the removable cartridge, low levels of formulation fluid, or low battery of an electronic device, e.g., a battery in removable electrical communication with the removable cartridge. In some embodiments, the perceivable signal may be a light, e.g., a green light indicating there are no errors or a red light indicating an error with the formula delivery system. In an example embodiment, the perceivable indicator would emit a red light signaling that the removable cartridge is improperly coupled to the first attaching structure. The light may be a solid or a blinking light, wherein, different frequencies of a blinking light may indicate different messages to the user. In other embodiments, the perceivable indicator is a speaker, and the perceivable signal is a sound transmitting from the speaker. The sound may be a simple beep or a pre-recorded vocal message to the user indicating that there is an error or specifying what the error is. The control module 630 is an electronic component responsible for managing the operation of the formulation delivery system. It may regulate various aspects of the device, including activation of the atomizer, sensor management, and dosage control. The control module may also provide error detection, alerting the user through visual or audio indicators if there are any issues with the system.

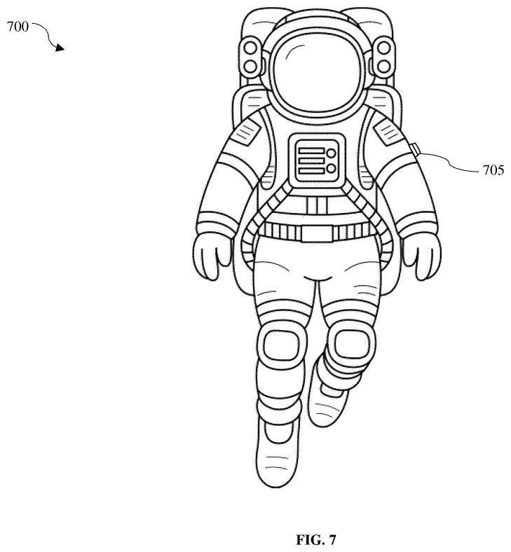

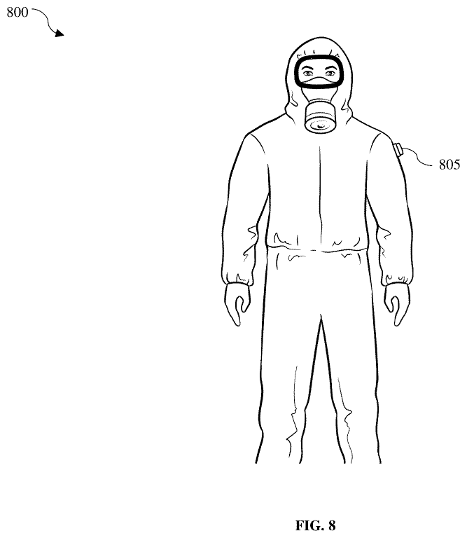

FIGS. 7-9 are exemplary embodiments of various at least partially enclosable wearable apparatus' comprising a first attaching structure. In FIG. 7 the at least partially enclosable wearable apparatus is a space suit 700 comprising a first attaching structure 705. The pace suit designed to integrate the formulation delivery system. In extreme environments like outer space, where maintaining controlled and sterile conditions is vital, this apparatus enables astronauts to receive medications without compromising the suit's integrity. The attaching structure is securely integrated into the space suit, likely positioned in a location that is easily accessible to the wearer, such as near the arm, helmet, or on a chest panel. The space suit is designed to maintain an airtight and isolated environment to protect the wearer from external elements, such as vacuum or hazardous particles in space. The attaching structure must incorporate air-tight seals and valves to ensure the medication delivery system does not compromise the suit's overall integrity while allowing medication to be delivered internally without exposure to the external environment. The valve system within the attaching structure ensures that medication can be released into the breathing environment of the space suit when needed. The valve would open upon proper engagement of the cartridge and close when the medication has been delivered, preserving the pressurized environment within the suit.

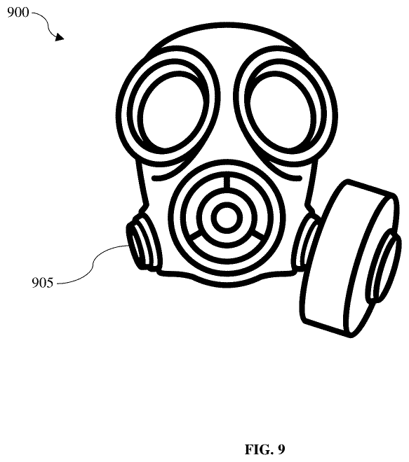

In FIG. 8 the at least partially enclosable wearable apparatus is a hazardous materials suit 800 comprising a first attaching structure 805. The hazardous materials (hazmat) suit is often used in environments with dangerous chemical, biological, or radioactive contaminants. The suit's primary function is to protect the wearer from exposure to these hazards while still enabling necessary medical treatments to be administered inside the suit. Similar to the space suit, the hazmat suit is equipped with a first attaching structure integrated into the suit. This structure allows the user to insert and engage the removable cartridge containing the required medication. The attaching structure in a hazmat suit must ensure that the user remains fully protected from the external environment while the medication is delivered. The hazmat suit's attaching structure features air-tight seals that maintain the integrity of the protective suit. These seals are critical to preventing any contaminants from entering the suit when the medication is being delivered. The seals engage automatically when the removable cartridge is inserted, ensuring that the wearer remains fully isolated from hazardous substances. The valve system in this configuration is designed to prevent any interaction between external contaminants and the medication delivery system. When the cartridge is inserted, the valve opens to release medication into the suit's breathing environment. Once the medication has been delivered, the valve seals the system to maintain the suit's air-tight protection.

In FIG. 9 the at least partially enclosable wearable apparatus is a respirator mask 900 comprising a first attaching structure 905. The respirator mask is typically used in both medical and industrial settings. Respirator masks protect the wearer from inhaling harmful particles, gases, or pathogens. In this configuration, the mask integrates the formulation delivery system to allow for direct medication delivery while still protecting the user's respiratory system from external pollutants. The respirator mask is equipped with a first attaching structure that interfaces with the removable cartridge. This structure could be positioned on the mask itself, close to the mouth or nose area, allowing for easy insertion of the cartridge and seamless delivery of the medication into the breathing chamber of the mask. The attaching structure must maintain the mask's breathability while ensuring that no external contaminants are introduced when the medication is delivered. The valve system within the mask allows air to flow through the respirator filter while keeping the medication delivery pathway separate to ensure the wearer inhales only clean, filtered air and the aerosolized medication. The valves in the respirator mask control the flow of air and medication within the system. Upon inserting the cartridge, the valve opens to release the medication into the inhalation pathway. Once the medication is delivered, the valve closes to preserve the protective integrity of the mask and ensure no external contaminants enter the system. This configuration is particularly valuable in medical settings where patients need respiratory treatments, such as during asthma attacks or COPD management, while also requiring protection from pathogens or pollutants. It can also be used in industrial settings where workers are exposed to hazardous materials and may need immediate medication delivery without removing their protective gear.

In some embodiments, other partially enclosable wearable apparatus' may be used. In an example embodiment, the partially enclosable wearable apparatus is a mouthpiece having a first attaching structure at one end. The mouthpiece apparatus is designed for comfortable oral use, ensuring a tight seal around the user's lips to maximize the effectiveness of medication delivery. The mouthpiece could be made from medical-grade silicone or plastic, ensuring it is soft enough for comfort but durable enough for repeated use. The mouthpiece could be ergonomically shaped to fit comfortably in the mouth, and it may include a slight curvature to match the natural shape of the oral cavity. It would be designed for hands-free use, allowing users to inhale without needing to manually hold the mouthpiece in place for the duration of the treatment. The mouthpiece would include a first attaching structure that interfaces with the removable cartridge of the formulation delivery system. This structure would be located at the base of the mouthpiece, where the cartridge is inserted and locked into place. The attaching structure would feature air-tight seals to prevent any medication leakage during inhalation and ensure that the aerosolized medication is delivered directly into the user's respiratory system. A one-way valve system could be integrated into the mouthpiece to ensure that air or medication flows only in one direction-toward the user. The valve would open when the user inhales and close when they exhale, preventing any backflow of air or contaminants into the system. This ensures that the medication remains in the mouthpiece until it is inhaled, enhancing the efficiency of the delivery process.

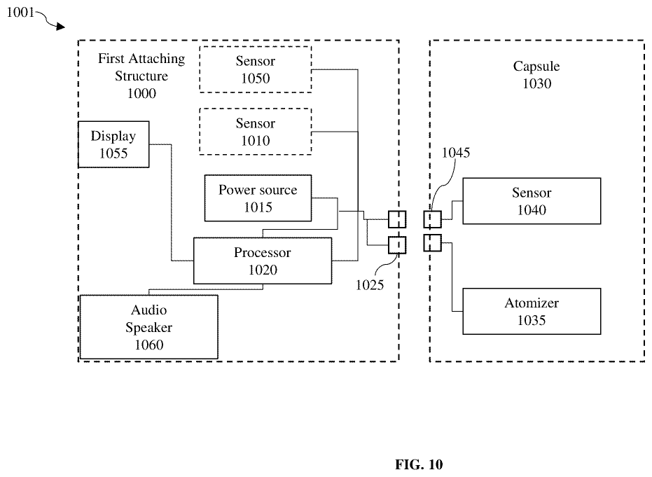

FIG. 10 is a diagram 1001 illustrating the main electrical components of the system for administering medication to a patient is shown, according to an example embodiment. Within a first attaching structure 1005, the sensor 1010, the power source 1015, the processor 1020, and a pair of electrical contacts 1025 are in electrical communication with each other. Additionally, within the capsule 1030, the atomizer 1035, the sensor 1040, and a pair of electrical connectors 1045 are in electrical communication with each other. When the two pairs of electrical contacts are contacting each other, the system provides electrical communication between the first attaching structure and the capsule, such that the power source can power the atomizer when the processor of the first attaching structure receives the signal to start the atomizer. The system may include a graphical display 1055 that is configured to provide visual instructions, warnings, maintenance items to the patient, such as when to start inhaling, when to stop inhaling, battery life of the device etc. when the device is in operation. The system may also include an audio component such as a speaker 1060 to provide audio instructions (that are similar to the instructions provided by graphical display) The system may also include a sensor 1050 for receiving audio commands from a user, such as when to start or stop the atomizer; however, other type of audio commands may be used and are within the spirit and scope of the present invention. In other embodiments, the first attaching structure may be used to connect to other types of devices.

It is understood that the device may include at least one sensor, or a plurality of sensors, consistent with this disclosure. These sensors may be implemented in various locations and configurations within the device to monitor and measure vital parameters, fluid dynamics, and operational states, thereby contributing to the precise control and safety of the medication administration. While specific examples of sensor types and their applications have been described, these are not meant to be limiting. The incorporation of sensors within the device can be adapted to suit various needs and may extend beyond the examples provided herein. Such variations and adaptations are contemplated to be within the spirit and scope of the present invention, highlighting the flexibility and comprehensiveness of the system's design in catering to a wide range of requirements and scenarios in administering medication to patients.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A formulation delivery system, comprising:
a removable cartridge comprising:
an atomizer disposed at a first end portion of the removable cartridge;
a wick comprising an absorbent material for absorbing a fluid, wherein one end of the wick abuts a portion of the atomizer such that the wick is configured to convey the fluid to the atomizer;
a channel defined by the removable cartridge, the channel extending from the portion of an outer surface to the wick; and
an electrical contact disposed at a second end portion of the removable cartridge.

2. The formulation delivery system of claim 1 further comprising:
a first attaching structure; and
the removable cartridge having a second attaching structure, wherein the second attaching structure is removably attached to the first attaching structure.

3. The formulation delivery system of claim 2 further comprising an at least partially enclosable wearable apparatus having the first attaching structure.

4. The formulation delivery system of claim 3 wherein the at least partially enclosable wearable apparatus comprises the first attaching structure comprising a receiving port configured to secure the removable cartridge, the receiving port including an air-tight seal to prevent environmental contamination of the fluid in the removable cartridge.

5. The formulation delivery system of claim 4, wherein the receiving port comprises a locking mechanism configured to engage with the removable cartridge, the locking mechanism being activated by inserting the removable cartridge into the receiving port.

6. The formulation delivery system of claim 5, wherein the locking mechanism is configured to include a sensor that detects a presence of the removable cartridge and provides a signal to initiate a fluid delivery.

7. The formulation delivery system of claim 6, wherein the sensor is selected from a group consisting of a pressure sensor, a magnetic sensor, and an optical sensor.

8. The formulation delivery system of claim 7, wherein the at least partially enclosable wearable apparatus further comprises a valve system, the valve system configured to open upon locking the removable cartridge in the receiving port and allowing controlled airflow through the at least partially enclosable wearable apparatus during the fluid delivery.

9. The formulation delivery system of claim 8 wherein the valve system comprises at least one of a check valve and an air lock disposed between the first attaching structure and the second attaching structure.

10. The formulation delivery system of claim 9, wherein the valve system comprises a seal of the removable cartridge to maintain environmental isolation within the wearable apparatus.

11. The formulation delivery system of claim 10, further comprising a control module disposed within the at least partially enclosable wearable apparatus, the control module being operatively connected to the atomizer to regulate a dosage, and a timing of a fluid delivery based on at least one predefined condition.

12. The formulation delivery system of claim 11, further comprising a perceivable indicator disposed on an external surface of the wearable apparatus, the perceivable indicator emits a perceivable signal indicating proper engagement of the removable cartridge.

13. The formulation delivery system of claim 12, wherein the perceivable indicator is further configured to provide a second perceivable signal of an error condition if the removable cartridge is improperly coupled to the first attaching structure.

14. The formulation delivery system of claim 13, wherein the at least partially enclosable wearable apparatus is an enclosed suit.

15. The formulation delivery system of claim 13, wherein the at least partially enclosable wearable apparatus is an enclosed mask.

16. The formulation delivery system of claim 13, wherein the at least partially enclosable wearable apparatus is a helmet.

17. A formulation delivery system comprising:
a first attaching structure;
a removable cartridge having a second attaching structure, wherein the second attaching structure is removably attached to the first attaching structure, the removable cartridge comprising:
an atomizer disposed at a first end portion of the removable cartridge;
a channel defined by the removable cartridge; and
an electrical contact disposed at a second end portion of the removable cartridge.

18. The formulation delivery system of claim 17, wherein the channel extends to a first end portion of the removable cartridge and a removable cap is disposed externally to the channel.

19. The formulation delivery system of claim 17. wherein the first attaching structure is disposed on at least one of a partially enclosed article of clothing, partially enclosed mask, a mouthpiece, and a base unit.
